(19) United States
(12) Patent Application Publication
(10) Pub. No.: US 2010/0092384 A1
(43) Pub. Date: Apr. 15, 2010

(54) MULTIFUNCTIONAL NANOPARTICLES AND COMPOSITIONS AND METHODS OF USE THEREOF

(75) Inventors: Ambika Bumb, Greer, SC (US); Martin W. Brechbiel, Annandale, VA (US); Peter Choyke, Bethesda, MD (US); Lars Fugger, Oxfordshire (GB); Peter James Dobson, Oxfordshire (GB)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignees: The jUnited States of America as represented by Secretary, Dept. of Health and Human Service, BETHESDA, MD (US); University of Oxford, Oxfordshire (GB)

(21) Appl. No.: 12/531,841

(22) PCT Filed: Mar. 17, 2008
(86) PCT No.: PCT/US08/57206

§ 371 Date: Dec. 8, 2009

Related U.S. Application Data
(60) Provisional application No. 60/907,085, filed on Mar. 19, 2007.

(51) Int. Cl. A61K 49/06; A61K 49/18; A61K 49/00

(52) U.S. Cl. 424/1.29; 424/9.6; 424/9.32; 424/9.4

(57) ABSTRACT

Provided is a multifunctional particle comprising: (a) an inner metallic core, (b) a biocompatible shell comprising an optical contrast agent embedded therein, and (c) a targeting biomolecule conjugated to the biocompatible shell through a multidentate ligand, wherein the multidentate ligand is chelated to an imaging agent. Also provided are compositions comprising the multifunctional particle and methods of using the multifunctional particle, including a method of diagnostic imaging and a method of treatment.

### CROSS-REFERENCE TO RELATED APPLICATIONS

This patent application claims the benefit of U.S. Provisional Patent Application No. 60/907,085, filed Mar. 19, 2007, which is incorporated by reference.

### BACKGROUND OF THE INVENTION

Targeted delivery of therapeutics is a major goal of pharmaceutical development. Accurate imaging of drugs permits confirmation that the drug is “hitting” the target. Though many techniques exist, few allow for in vivo imaging and control of drug release at the cellular level. In the past two decades, studies using ultra-small superparamagnetic iron oxide nanoparticles (USPIOs) have provided a new potential technology to enhance molecular and cellular imaging. There are a number of SPIO compounds already approved for use in the clinic and others are in clinical trials, but most nonspecifically localize by exploiting the body's natural uptake. Rarely are the particles attached to ligands to target delivery to specific locations.

Technologies such as optical imaging have the advantage of high spatial and temporal resolution but have limited depth penetration due to light diffusion through tissue. Imaging of radioisotopes using single photon emission computed tomography (SPECT) is useful for quantification purposes but it lacks spatial and temporal resolution. Magnetic resonance imaging (MRI) is a powerful tool for clinicians; however, this technique lacks sensitivity.

Thus, there exists a need for multi-imageable nanoparticle bioconjugates as sensitive and versatile probes for in vivo cellular and molecular imaging.

### BRIEF SUMMARY OF THE INVENTION

The invention provides a nanoparticle that is imageable by three separate and distinct properties through magnetic resonance (MR), optical, and radioisotope imaging. In particular, the invention provides a multifunctional particle comprising: (a) an inner metallic core, (b) a biocompatible shell comprising an optical contrast agent embedded therein, and (c) a targeting biomolecule conjugated to the biocompatible shell and a multidentate ligand, wherein the multidentate ligand is chelated to an imaging agent. The multifunctional particle utilizes three imaging techniques providing a more effective diagnostic tool. For example, a magnetic nanoparticle that is labeled by both a radioisotope and an optical contrast agent allows for high resolution imaging and quantification with the ability to verify that the particle has reached its target through three images. For in vitro studies, having a fluorescent agent provides ease for use with typical analysis tools such as confocal microscopy and flow cytometry, whereas the magnetic properties allows for ease of separation by use of a magnet.

A composition comprising at least one multifunctional particle; and a carrier is also provided.

A method for diagnostic imaging in a host is further provided. The method comprises administering to the host a multifunctional particle, in an amount effective to provide an image; and exposing the host to an energy source, whereupon a diagnostic image is obtained.

Still further provided is a method for treating a cellular disorder in a mammal. The method comprises administering to the mammal a multifunctional particle in an amount effective to treat the cellular disorder, whereupon the cellular disorder in the mammal is treated.

### BRIEF DESCRIPTION OF THE SEVERAL VIEWS OF THE DRAWING(S)

FIG. 1 depicts a multifunctional particle (10), in which an inner metallic core (1) is coated with a biocompatible shell (2) which can comprise an inner shell (2a) and an outer shell (2b), and which comprises an optical contrast agent (3) embedded therein, and which a targeting biomolecule (4) is conjugated to the biocompatible shell (2) and a multidentate ligand (5) that is chelated to an imaging agent (6).

FIG. 2 illustrates the coupling of a nanoparticle to a targeting biomolecule. An antibody is coupled to a bifunctional crosslinker, sulfosuccinimidyl-4-(N-maleimidomethyl)cyclohexane-1-carboxylate (s-SMCC). The biocompatible shell of the multifunctional particle has been functionalized with (3-mercaptopropyl)trimethoxysilane (MPS) to provide a thiol-activated nanoparticle (NP). The maleimide-activated antibody can be coupled to the thiol-activated NP.

FIG. 3 illustrates the coupling of a nanoparticle to a targeting biomolecule. An antibody is coupled to s-SMCC, which is then reacted with MPS. The activated antibody is then coupled to the biocompatible shell of an NP.

FIG. 4A illustrates the coupling of 3-aminopropyltriethoxysilane (APTES)) and s-SMCC, which is then conjugated to the biocompatible shell of an NP. FIG. 4B illustrates the coupling of an antibody to 2-(p-isothiocyanatobenzyl)-cyclohexyl-diethylenetriaminepentaacetic acid (“CHXA″”). The antibody is treated with Traut's reagent to form free thiol groups. The activated antibody is then coupled to the maleimide-activated NP.

### DETAILED DESCRIPTION OF THE INVENTION

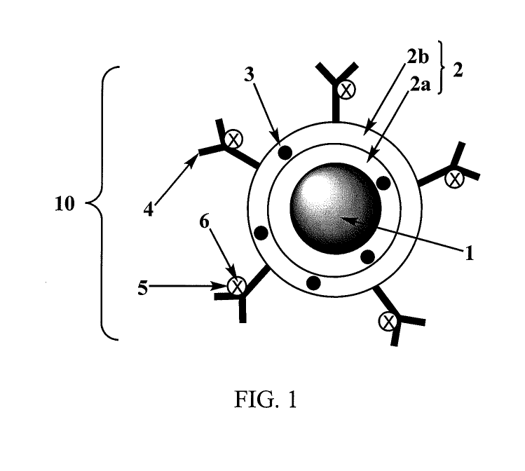

The invention provides a multifunctional particle comprising: (a) an inner metallic core, (b) a biocompatible shell comprising an optical contrast agent embedded therein, and (c) a targeting biomolecule conjugated to the biocompatible shell and a multidentate ligand, wherein the multidentate ligand is chelated to an imaging agent. For example, FIG. 1 illustrates a multifunctional particle (10) comprising an inner metallic core (1), a biocompatible shell (2), which can comprise an inner shell (2a) and an outer shell (2b), and comprising an optical contrast agent (3) embedded therein, and a targeting biomolecule (4) conjugated to the biocompatible shell (2) and a multidentate ligand (5), wherein the multidentate ligand is chelated to an imaging agent (6).

The particles can provide in vivo imaging for verification of location and quantification of the delivered structure. An optical and MR imageable particle is useful for in vitro purposes, but attaching a radioisotope as a third mode of imaging provides advantages for quantification of delivered construct and biodistribution studies in vivo. Furthermore, targeting these particles would create a noninvasive reporting tool used to monitor a variety of specific biological responses while providing valuable information regarding physiology and pathophysiology.

The multifunctional particle comprises an inner metallic core (depicted as 1 in FIG. 1). The metallic core is made from any suitable metal or metal alloy that forms nanoparticles (e.g., cobalt, iron, iron-cobalt, copper, platinum, nickel, gold, silver, titanium, ruthenium, and alloys thereof). Typically the nanoparticle has a well-defined and regular shape and has a narrow size distribution (i.e., is monodisperse). Preferably, the inner metallic core is magnetic (e.g., iron, nickel, cobalt, and alloys thereof).

In an especially preferred embodiment, the inner metallic core comprises superparamagnetic iron oxide, such as maghemite/magnetite (γ-Fe₂O₃/Fe₃O₄). Preferably, the metallic core is an ultra-small superparamagnetic iron oxide nanoparticle (USPIO).

For the multifunctional particles of the invention, the diameter of the inner metallic core is typically less than about 50 nm on average (e.g., about 1 nm to about 40 nm, about 5 nm to about 25 nm, less than about 15 nm, about 9 nm, on average). The diameter typically can be controlled based on reaction parameters. Preferably, the diameter of the nanoparticle is selected based on desired end use properties, e.g., the particles are small enough to circulate without being rapidly removed by the reticuloendothelial system.

The metallic cores can be purchased (e.g., Strem Chemicals, Newburyport, Mass.) or synthetically prepared. There are several methods to synthesize nanoparticles, particularly monodisperse nanoparticles. For example, such methods include coprecipitation of metal salts (Shen et al., Magnetic Resonance in Medicine 29, 599-604 (1993); Kim et al., Chemistry of Materials 15, 4343-4351 (2003)), reverse micelle synthesis (Pileni et al., Nature Materials 2, 145-150 (2003); Seip et al., Nanostructured Materials 12, 183-186 (1999)), attrition, pyrolysis, thermolysis, or polyol- or alcohol-reduction methods.

In a specific example, co-precipitation of ferrous and ferric salts in alkaline and acidic aqueous phases can be used to prepare colloids of Fe₃O₄ nanoparticles in the size range of 10-20 nm (Massart et al., IEEE Transactions on Magnetics 17, 1247-1248 (1981)). Temperature, ionic strength, pH, and the presence of other ions can be manipulated to alter the size of particles produced (Vayssieres et al., Journal of Colloid and Interface Science 205, 205-212 (1998)).

The inner metallic core is coated with a biocompatible shell (depicted as 2 in FIG. 1) to prevent clearance of the particles, to reduce aggregation of metallic cores, and/or to prevent absorbance of fluorescence by the metallic core. Thus, the biocompatible shell is prepared from any material that can be linked to both the metallic inner core and the biomolecule and enable the multifunctional particle to maintain its in vivo utility. Suitable materials include, for example, silica, polyethylene glycol (PEG), dextran, and dimercaptosuccinic acid (DMSA). The biocompatible shell can comprise two layers: a first innermost layer shell (depicted as 2a in FIG. 1) that is in contact with (e.g., bonded to) the inner metallic core and a second outermost layer shell (depicted as 2b in FIG. 1). The first innermost and second outermost layers of the biocompatible shell can be prepared from the same or different material. While the illustrated embodiments show the biocompatible shell as two layers, it is to be understood that when the first innermost and second outermost shells are prepared from the same material, typically a single layer is produced in the resulting particle.

The total thickness of the biocompatible shell typically is less than about 10 nm, preferably about 5 nm or less, and more preferably between about 1 nm and 5 nm. The thickness of the second outermost layer typically is about 0.5 nm to about 3 nm, and preferably about 2.5 nm.

In a preferred embodiment, both the first innermost and second outermost layers of the biocompatible shell comprise silica. Silica shells can be formed from various starting materials, including tetraethylorthosilicate (TEOS). Silica is well known for its optical transparency (Liu et al., Acta Materialia 47, 4535-4544 (1999)), and the advantage it offers for this application is its tunable thickness. The surface of silica can be coated with silanol groups that easily react with alcohols and silane coupling agents (Ulman et al., Chem. Rev. 96, 1533-1554 (1996)) to produce dispersions that are stable in non-aqueous solvents and are ideal for strong covalent bonding with ligands. The silica shell would also play a role in maintaining stability for particle suspensions during changes in pH or electrolyte concentration, due to silanol groups that make the surface lyophilic (Mulvaney et al., J. Mater. Chem. 10, 1259-1270 (2000)).

One method to prepare silica shells is the Stöber method (Journal of Colloid and Interface Science 26, 62-69 (1968)). Briefly, the process involves hydrolysis of an alkoxy silane and condensation of alcohol and water (Bardosova et al., Journal of Materials Chemistry 12, 2835-2842 (2002)).

The biocompatible shell comprises at least one contrast agent (depicted as 3 in FIG. 1). The contrast agent can be bonded anywhere within the shell, including the first innermost layer, the second outermost layer, or both. To bond the contrast agent, the biocompatible shell can be reacted with a linking group to covalently link the contrast agent to the surface of the first innermost layer, the second outermost layer, or both. The linking group is any organic molecule that can react with both the biocompatible shell materials (e.g., a silanol group) and the contrast agent. An example of a linking group is 3-aminopropyltriethoxysilane. Subsequent to conjugation of the contrast agent, an additional layer of the biocompatible shell (e.g., silica) can be deposited to entrap the dye, ensure biocompatibility, and provide a surface for biomolecule conjugation.

The contrast agent embedded in the biocompatible shell can be any moiety that generates UV-Vis radiation only when excited by a source of radiation having a wavelength different from the emitted wavelength. For example, the contrast agent can be a cyanine dye, rhodamine, coumarin, pyrene, dansyl, fluorescein, fluorescein isothiocyanate, carboxyfluorescein diacetate succinimidyl ester, an isomer of fluorescein, R-phycoerythrin, tris(2′,2-bipyridyl)dichlororuthenium(II) hexahydrate, Fam, VIC®, NED™, ROX™, calcein acetoxymethylester, DiIC₁₂, or anthranoyl.

In a preferred embodiment, the contrast agent is a cyanine dye. The cyanine dye can be, for example, Cy5.5, Cy5, or Cy7 (GE Healthcare, Chalfont St Giles, Buckinghamshire, UK). Preferably, the contrast agent is Cy5.5:

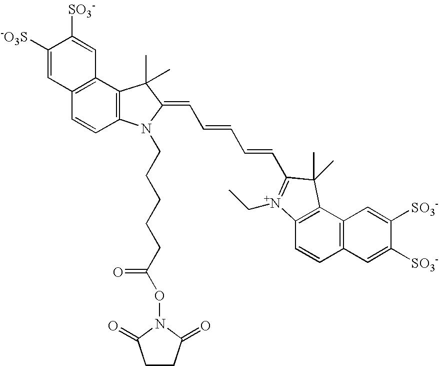

Cy5.5 has excitation and emission peaks at 675 nm and 694 nm, respectively. It is a highly sensitive and bright dye with high extinction coefficients and favorable quantum yields. It has superior photostability compared to more commonly used dyes allowing more time for image detection. Cy5.5 is a good candidate for physiological use because it is stable in the pH range of 3 to 10, soluble in aqueous and organic solvents, and has low non-specific binding.

Cy5.5 is commercially available with an N-hydroxysuccinimide (NHS) ester group for binding to amine groups. Thus, a linker comprising a free amino group (e.g., 3-aminopropyltriethoxysilane (APTES)) can be used to conjugate Cy5.5 to the particle. The free amine of the linker can bind to the active NHS ester of Cy5.5, as illustrated in the following reaction scheme:

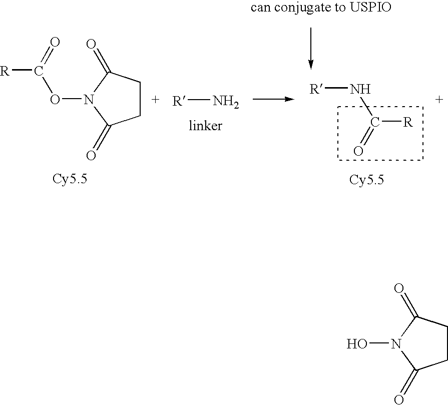

In the case of a silane-containing linker, such as APTES, the silane groups can attach to the particle surface using known procedures (e.g., the Stöber mechanism).

The biocompatible shell is conjugated to a targeting biomolecule (depicted as 4 in FIG. 1), which, in turn, is conjugated to a multidentate ligand (depicted as 5 in FIG. 1). The term “biomolecule” refers to all natural and synthetic molecules that play a role in biological systems. A biomolecule includes a hormone, an amino acid, a peptide, a peptidomimetic, a protein, deoxyribonucleic acid (DNA), ribonucleic acid (RNA), a lipid, an albumin, a polyclonal antibody, a receptor molecule, a receptor binding molecule, a hapten, a monoclonal antibody (i.e., an immunoglobulin), and an aptamer. Specific examples of biomolecules include insulins, prostaglandins, growth factors, liposomes and nucleic acid probes. An advantage of using biomolecules is tissue targeting through specificity of delivery. In a preferred embodiment, the targeting biomolecule is an antibody (e.g., scFv, F(ab′)₂, and Fab), a peptide, or a protein. Specific antibodies include, for example, a single chain antibody (scAb), a scAb to c-erbB-2, L243, C46 Ab, 85A12 Ab, H17E2 Ab, NR-LU-10 Ab, HMFCl Ab, W14 Ab, RFB4 Ab to B-lymphocyte surface antigen, A33 Ab, TA-99 Ab, trastuzumab (e.g., Herceptin™) and cetuximab (e.g., Erbitux™, ImClone and Bristol-Myers-Squibb).

Linkage analyses and association studies have shown that susceptibility to multiple sclerosis (MS) is associated with genes in the human histocompatibility leukocyte antigens (HLA) class II region. L243 is an anti-HLA-DR monoclonal antibody (mAb) that can be used to direct the nanoparticles to the inflammatory foci in the brain for MS. In an embodiment, nanoparticles can be conjugated to L243 to image cells that express HLA (e.g., HLA-DR). A DR2-expressing humanized mouse model is available for studies for MS (Lang et al., Nat. Immunol. 3, 940-943 (2002); Madsen et al., Nat. Genet. 23, 343-347 (1999)).

HER2 is a membrane bound receptor associated with tyrosine kinase activity that is over-expressed in a variety of epithelial cancers, including breast, ovarian, pancreatic, and colorectal carcinomas (Milenic et al., Clinical Cancer Research 10, 7834-7841 (2004)), making it an ideal target for therapy (Natali et al., Int. J. Cancer 45, 457-461 (1990)). Trastuzumab is a humanized mAb that targets HER2 on epithelial cancer cells. Trastuzumab is commercially available from Genentech as Herceptin™. In an embodiment, NPs can be conjugated to Herceptin™ to image cancer cells that over-express HER2.

One method to test whether the attached Ab will successfully carry the nanoparticle (NP) to its target is to stain cells with the Ab-NP conjugate and analyze them with flow cytometry. If the Ab was successful in tagging cells with NPs, the cells would fluoresce. For example a nanoparticle comprising Cy5.5 would fluoresce with near infrared emissions.

Several methods are known in the art to conjugate a biomolecule to a biocompatible shell of a metallic nanoparticle. See, e.g., Wolcott et al., Journal of Physical Chemistry B 110, 5779-5789 (2006); Lu et al., Analytical Chemistry 67, 83-87 (1995); Zhao et al., Proceedings of the National Academy of Sciences of the United States of America 101, 15027-15032 (2004); Santa et al., Analytical Chemistry 73, 4988-4993 (2001); Yang et al., Analyst 128, 462-466 (2003); Wang et al., Nano Letters 5, 37-43 (2005); and Jonsson et al., Biochemical Journal 227, 363-371 (1985).

For example, a bifunctional linker can be used, such as a heterobifunctional linker or a homobifunctional linker. Suitable bifunctional linkers comprise reactive moieties, such as a succinimidyl ester, a maleimide, or iodoacetamide. Suitable specific bifunctional linkers include sulfosuccinimidyl-4-(N-maleimidomethyl)cyclohexane-1-carboxylate (s-SMCC), sulfosuccinimidyl 4-(N-maleimidomethyl)cyclohexane-1-carboxylate (sulfo-SMCC), succinimidyl-4-[N-maleimidomethyl]cyclohexane-1-carboxy-[6-amidocaproate] (LC-SMCC), N-hydroxysuccinimide (NHS), N-hydroxysulfosuccinimide (sulfo-NHS), succinimidyl 3-(2-pyridyldithio)propionate (SPDP), succinimidyl 6-(3-[2-pyridyldithio]-propionamido)hexanoate (LC-SPDP), succinimidyl-6-[β-maleimidopropionamido]hexanoate (SMPH), succinimidyl 4-maleimidobutyrate (GMBS), N-[g-maleimidobutyryloxy]sulfosuccinimide ester (sulfo-GMBS), succinimidyl 6-maleimidocaproate (EMCS), N-e-maleimidocaproyloxy]sulfosuccinimide ester (sulfo-EMCS), succinimidyl-4-(p-maleimidophenyl)butyrate (SMPB), sulfosuccinimidyl 4-[p-maleimidophenyl]butyrate (sulfo-SMPB), 1-ethyl-3-[3-dimethylaminopropyl]carbodiimide hydrochloride (EDC), m-maleimidobenzoyl-N-hydroxysulfosuccinimide ester (MBS), m-maleimidobenzoyl-N-hydroxysulfosuccinimide ester (sulfo-MBS), N-k-maleimidoundecanoic acid (KMUA), N-e-maleimidocaproic acid (EMCA), N-succinimidyl iodoacetate (SIA), N-succinimidyl[4-iodoacetyl]aminobenzoate (SIAB), N-sulfosuccinimidyl[4-iodoacetyl]aminobenzoate (sulfo-SIAB), succinimidyl 3-[bromoacetamido]propionate (SBAP), bismaleimidohexane (BMHH), tris[2-maleimidoethyl]amine (TMEA), 1,6-hexane-bis-vinylsulfone (HBVS), disuccinimidyl suberate (DSS). Other bifunctional linkers are known in the art and are commercially available from, e.g., Pierce Chemical Co. (Rockford, Ill.).

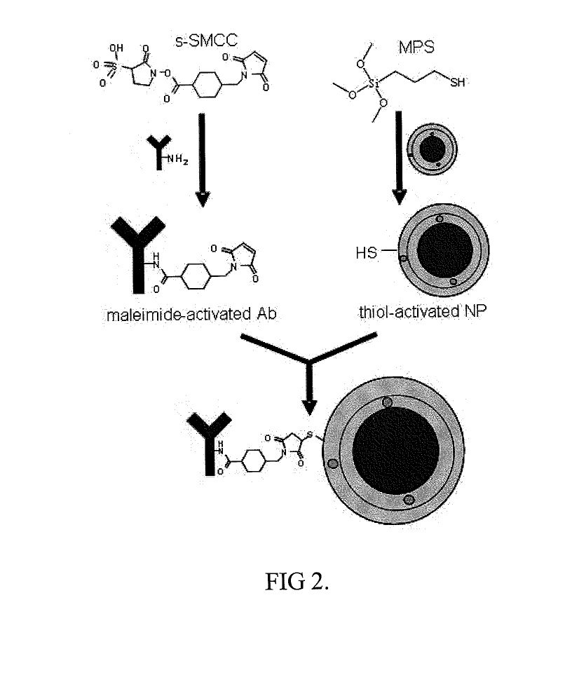

Preferably, the bifunctional linker is s-SMCC, which is a water-soluble and non-cleavable crosslinker that contains an amine-reactive NHS ester and a sulfhydryl-reactive maleimide group. Amines on an antibody (Ab) or protein form strong amide bonds with the NHS ester of s-SMCC (Wolcott et al., Journal of Physical Chemistry B 110, 5779-5789 (2006)). See FIG. 2. The surface of the biocompatible shell can be functionalized with thiols using a (3-mercaptopropyl)trimethoxysilane (MPS), by, for example, the Stöber mechanism. The double bond of s-SMCC's maleimide undergoes an alkylation reaction with free NP thiol groups to form stable thioether bonds.

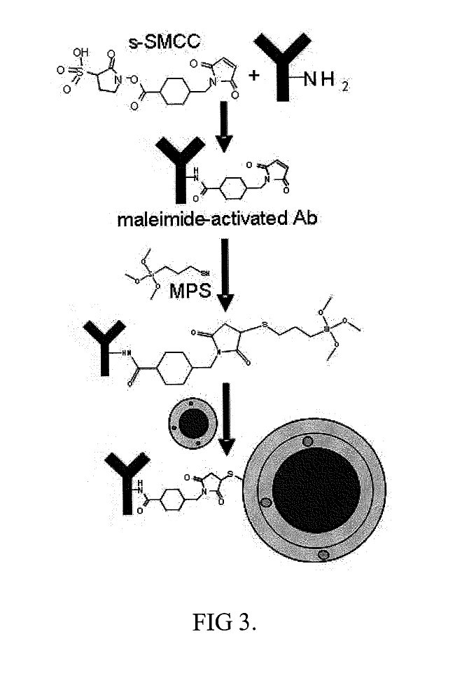

Alternatively, s-SMCC can reacted with a free amino group on the biomolecule, such as an antibody. The maleimide-activated antibody can reacted with MPS, which in turn can react with the biocompatible shell of the metallic nanoparticle. See FIG. 3.

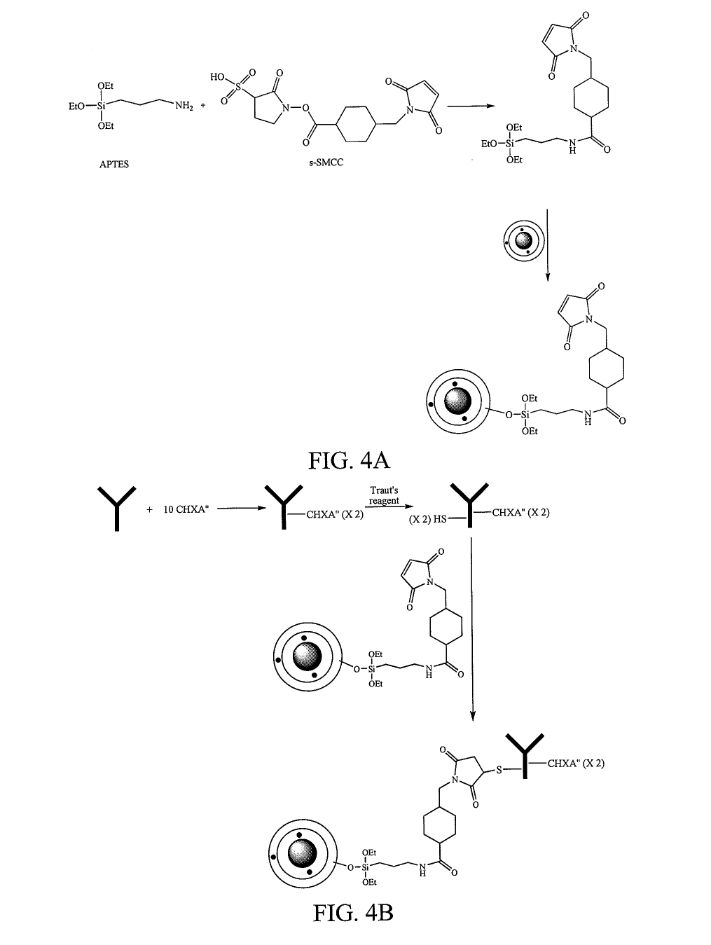

The biocompatible shell of the metallic nanoparticle also can be functionalized with a linker based on 3-aminopropyltriethoxysilane (APTES)) and s-SMCC (FIG. 4A). The maleimide-activated NP can be conjugated to a free thiol group on a biomolecule, such as an antibody, that is optionally conjugated to a multidentate ligand, discussed below (FIG. 4B).

The biomolecule is conjugated to a multidentate ligand. The multidentate ligand is any ligand that can chelate a metal and be covalently bound to both the biocompatible shell and the biomolecule. Typically the multidentate ligand is selected based on the coordination chemistry of the chosen radionuclide. For example, the multidentate ligand can be based on diethylenetriaminepentaacetic acid (“DTPA”), 1,4,7-triazacyclononane-N,N′,N″-triacetic acid (“NOTA”), or 1,4,7,10-tetraazacyclododecane-N,N′,N″,N′″-tetraacetic acid (“DOTA”).

Multidentate ligands based on DTPA include 2-(p-aminobenzyl)-6-methyl-1,4,7-triaminoheptane-N,N′,N″-pentaacetic acid (“1B4M-DTPA”) and 2-(p-isothiocyanatobenzyl)-cyclohexyl-diethylenetriaminepentaacetic acid (“CHX-DTPA”). In some embodiments, the multidentate ligand can be based on CHX-DTPA:

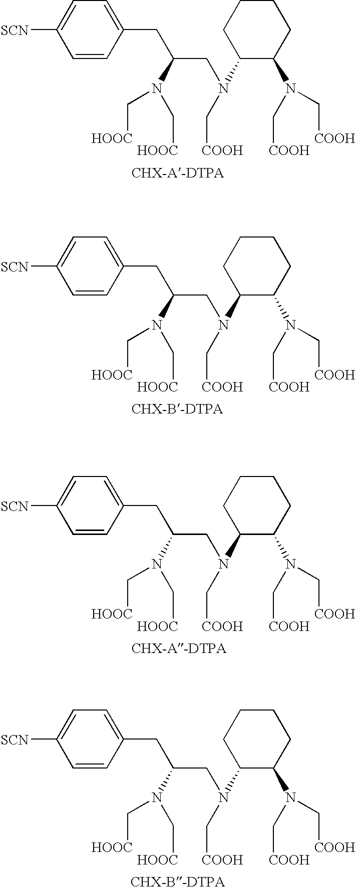

The aromatic isothiocyanate arms on the benzyl group can be used for attaching to a reactive moiety (e.g., an amine) on biomolecules, such as antibodies or proteins.

Several bifunctional derivatives of DOTA are known, including 2-(p-aminobenzyl)-1,4,7,10-tetraazacyclododecane-N,N′,N″,N′″-tetracarboxamide (“TCMC”), 2-(p-isothiocyanatobenzyl)-1,4,7,10-tetraazacyclododecane-N,N′,N″,N′″-tetraacetic acid (“C-DOTA”), and 1,4,7,10-tetraaza-N-(1-carboxy-3-(4-nitrophenyl)propyl)-N′,N″,N′″-tris(acetic acid) cyclododecane (“PA-DOTA”):

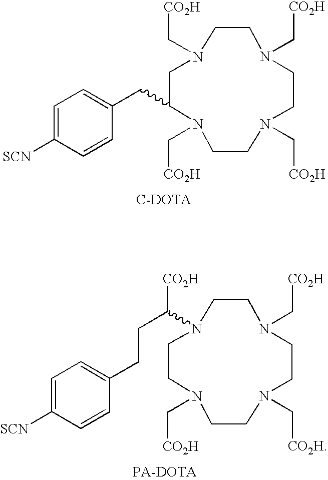

Other suitable DOTA derivatives include those that that are backbone-substituted. For example, the multidentate ligand can be a compound of formula (I), (II), or (III):

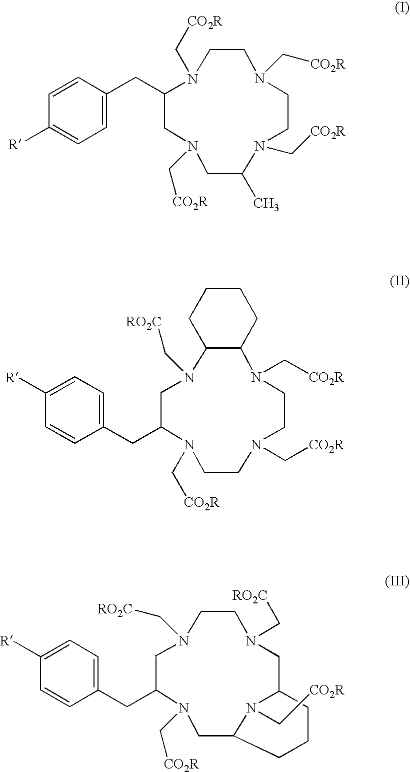

wherein R is hydrogen or alkyl and R′ is selected from the group consisting of hydrogen, halo, alkyl, hydroxy, nitro, amino, alkylamino, thiocyano, isothiocyano, carboxyl, carboxyalkyl, carboxyalkyloxy, amido, alkylamido, and haloalkylamido.

Additional examples of suitable multidentate ligands are described in, for example, U.S. Pat. Nos. 7,163,935, 7,081,452, 6,995,247, 6,765,104, 5,434,287, 5,286,850, 5,246,692, 5,124,471, 5,099,069, and 4,831,175 and U.S. Patent Application Publication No. 2006/0165600.

Coupling of a multidentate ligand to one or more biomolecules can be accomplished by several known methods (see, for example, Krejcarek et al., Biochem. Biophys. Res. Commun., 30, 581 (1977); and Hnatowich et al., Science, 220, 613 (1983)). For example, a reactive moiety present in a backbone or sidechain substituent (e.g., isothiocyanato) is coupled with a second reactive group located on the biomolecule. Typically, a nucleophilic group is reacted with an electrophilic group to form a covalent bond between the biomolecule and the multidentate ligand. Examples of nucleophilic groups include amines, anilines, alcohols, phenols, thiols, and hydrazines. Examples of electrophilic groups include halides, disulfides, epoxides, maleimides, acid chlorides, anhydrides, mixed anhydrides, activated esters, imidates, isocyanates, and isothiocyanates.

Preferably, the backbone or sidechain substituent on the multidentate ligand is a substituent that conjugates the compound to an antibody. This substituent is desirably a free-end nitro group, which can be reduced to an amine. The amine then can be activated with a compound, such as thionyl chloride, to form a reactive chemical group, such as an isothiocyanate. An isothiocyanate is preferred because it links directly to an amino residue of an antibody, such as an mAb. The aniline group can be linked to an oxidized carbohydrate on the protein and, subsequently, the linkage fixed by reduction with cyanoborohydride. The amino group also can be reacted with bromoacetyl chloride or iodoacetyl chloride to form —NHCOCH₂Z, with Z being bromide or iodide. This group reacts with any available amine or sulfhydryl group on a biomolecule to form a stable covalent bond. The most desirable backbone or sidechain substituents for multidentate ligands are members selected from the group consisting of hydrogen, halo, alkyl, hydroxy, nitro, amino, alkylamino, thiocyano, isothiocyano, carboxyl, carboxyalkyl, carboxyalkyloxy, amido, alkylamido and haloalkylamido. In some preferred instances, the backbone or sidechain substituent is a haloalkylamido of the formula —NHCOCH₂Z, with Z being bromide or iodide. Another preferred substituent for this position is isothiocyano (—NCS).

For conjugation, the biomolecule (e.g., antibody or protein) is prepared at a suitable concentration and in an appropriate buffer. It is then reacted with the multidentate ligand, after which, the product is purified. The solvent of the immunoconjugate must then be changed to a buffer suitable for radiolabeling, and subsequent injection or storage. An important requirement for the entire process is that it is conducted under stringent metal-free conditions. Typically, all vessels and reagents are prepared to meet this constraint.

The multidentate ligand is complexed to an imaging agent that is optionally radioactive. The imaging agent is any metal ion that is suitable for the desired end use of the multifunctional particle. For example, in proton magnetic resonance imaging, paramagnetic metal atoms such as gadolinium(III), manganese(II), manganese(III), chromium(III), iron(II), iron(III), cobalt(II), nickel(II), copper(II), praseodymium(III), neodymium(III), samarium(III), ytterbium(III), terbium(III), dysprosium(III), holmium(III), and erbium(III) (all are paramagnetic metal atoms with favorable electronic properties) are preferred as metals complexed by the multidentate ligand. Gadolinium(III) is the most preferred complexed metal due to the fact that it has the highest paramagnetism, low toxicity when complexed to a suitable ligand, and high lability of coordinated water. Typical metal ions for forming a complex of the invention include Ac, Bi, Pb, Y, Mn, Cr, Fe, Co, Ni, Tc, In, Ga, Cu, Re, a lanthanide (i.e., any element with atomic number 57 to 71 inclusive) and an actinide (i.e., any element with atomic number 89 to 103 inclusive). For use as x-ray contrast agents, the metal ion must be able to absorb adequate amounts of x-rays (i.e., radio-opaque), such as, for example, indium, yttrium, lead, bismuth, gadolinium, dysprosium, holmium and praseodymium.

The multidentate ligand also can be complexed with a radioactive metal ion. Radioisotopes of any suitable metal ion are acceptable for forming metal complexes of the invention. For example, typical radioisotopes include technetium, bismuth, lead, actinium, nitrogen, iodine, fluorine, tellurium, helium, indium, gallium, copper, rhenium, yttrium, samarium, zirconium, iodine, and holmium. Of these radioisotopes, indium is preferred. Specific examples of radionuclides suitable for complexing to a multidentate ligand for various imaging techniques, including single photon emission computed spectroscopy, are, for example, ²¹³Bi, ²¹²Bi, ²¹²Pb, ²⁰³Pb, ²²⁵Ac, ¹⁷⁷Lu, ^{99m}Tc, ¹¹¹In, ¹²⁴I, ¹²³I, ¹⁸⁶Re, ²⁰¹Tl, ³He, ¹⁶⁶Ho, ⁸⁶Y, ⁶⁴Cu, ⁸⁹Zr, ⁶⁶Ga, ⁶⁸Ga, and ⁶⁷Ga. The radioisotope ¹¹¹In is especially preferred.

In a preferred embodiment, the imaging agent is a radioisotope, preferably a gamma-emitting radioisotope. The gamma-emitting radioisotope can be, for example, a radioactive lanthanide. Specific radioisotopes that are preferred include ⁸⁶Y, ⁶⁴ Cu, 89Zr, ¹²⁴I, ⁶⁶Ga, ⁶⁸Ga, ⁶⁷Ga, ¹²³I, ²⁰³Pb, and ¹¹¹In.

To prepare metal complexes of the invention, the multidentate ligand-NPs are complexed with an appropriate metal or metal ion. This can be accomplished by any methodology known in the art. For example, the metal can be added to water in the form of an oxide, halide, nitrate or acetate (e.g., yttrium acetate, bismuth iodide) and treated with an equimolar amount of multidentate ligand. The multidentate ligand can be added as an aqueous solution or suspension. Dilute acid or base can be added (where appropriate) to maintain a suitable pH. Heating at temperatures as high as 100° C. for periods of up to 24 hours or more can be employed to facilitate complexation, depending on the metal, the multidentate ligand, and their concentrations.

The invention further provides a composition comprising (a) at least one multifunctional particle according to an embodiment of the invention; and (b) a carrier. In some embodiments, the carrier can be pharmaceutically acceptable. Pharmaceutically acceptable carriers, for example, vehicles, adjuvants, excipients, and diluents, are well-known to those ordinarily skilled in the art and are readily available to the public. The choice of carrier will be determined, in part, by the particular composition and by the particular method used to administer the composition. Accordingly, there is a wide variety of suitable formulations of the pharmaceutical compositions of the present invention.

Suitable formulations include aqueous and non-aqueous solutions, isotonic sterile solutions, which can contain anti-oxidants, buffers, bacteriostats, and solutes that render the formulation isotonic with the blood or other bodily fluid of the intended recipient, and aqueous and non-aqueous sterile suspensions that can include suspending agents, solubilizers, thickening agents, stabilizers, and preservatives. In one embodiment, the pharmaceutically acceptable carrier is a liquid that contains a buffer and a salt. The formulation can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets.

Further carriers include sustained-release preparations, such as semipermeable matrices of solid hydrophobic polymers containing the active agent, which matrices are in the form of shaped articles (e.g., films, liposomes, or microparticles).

The pharmaceutical composition can include thickeners, diluents, buffers, preservatives, surface active agents, and the like. The pharmaceutical compositions can also include one or more additional active ingredients, such as antimicrobial agents, anti-inflammatory agents, anesthetics, and the like.

The pharmaceutical composition comprising the multifunctional particle can be formulated for any suitable route of administration, depending on whether local or systemic treatment is desired, and on the area to be treated. Desirably, the pharmaceutical composition is formulated for parenteral administration, such as intravenous, intraperitoneal, intraarterial, intrabuccal, subcutaneous, or intramuscular injection. In a preferred embodiment, the multifunctional particle or a composition thereof is administered intravenously.

Injectables can be prepared in conventional forms, either as liquid solutions or suspensions, solid forms suitable for suspension in liquid prior to injection, or as emulsions. Additionally, parental administration can involve the preparation of a slow-release or sustained-release system, such that a constant dosage is maintained. Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The pharmaceutical composition also can be administered orally. Oral compositions can be in the form of powders or granules, suspensions or solutions in water or non-aqueous media, capsules, sachets, or tablets. Thickeners, flavorings, diluents, emulsifiers, dispersing aids, or binders may be desirable.

Suitable carriers and their formulations are further described in A. R. Gennaro, ed., Remington: The Science and Practice of Pharmacy (19th ed.), Mack Publishing Company, Easton, Pa. (1995).

The dose administered to a mammal, particularly a human, in the context of the present invention should be sufficient to effect a therapeutic response in the mammal over a reasonable time frame or an amount sufficient to allow for diagnostic imaging of the desired tissue or organ. The dose will be determined by the strength of the particular compositions employed and the condition of the mammal (e.g., human), as well as the body weight of the mammal (e.g., human) to be treated. The size of the dose also will be determined by the existence, nature, and extent of any adverse side effects that might accompany the administration of a particular composition. A suitable dosage for internal administration is 0.01 to 100 mg/kg of body weight per day, such as 0.01 to 35 mg/kg of body weight per day or 0.05 to 5 mg/kg of body weight per day. A suitable concentration of the compound in pharmaceutical compositions for topical administration is 0.05 to 15% (by weight), preferably 0.02 to 5%, and more preferably 0.1 to 3%.

A method for obtaining a diagnostic image in a mammal is provided by the present invention. In particular, an embodiment of the method comprises administering to the mammal a multifunctional particle of the invention, in an amount effective to provide an image; and exposing the mammal to an energy source, whereupon a diagnostic image in the mammal is obtained. The diagnostic image can be, for example, a magnetic resonance image (MRI), an x-ray contrast image, single photon emission computed spectroscopy (SPECT) image, positron emission tomography (PET) image, or the like.

The method can be used to image cells, such as cancer cells, in the mammal. One embodiment of the method comprises (a) administering to a mammal intravenously a multifunctional particle of the invention; (b) contacting a cancer cell surface receptor with the targeting biomolecule of the particle; and (c) observing a fluorescence emission from the optical contrast agent or detecting an emission from the imaging agent by spectroscopy. The spectroscopy can be, for example, SPECT, PET, gamma scintigraphy, or MRI. Preferably, the targeting biomolecule binds to a receptor on the surface of a cancer cell.

The cells are preferably cancer cells, more preferably cancer cells that over-express HER1 and/or HER2. The human epidermal growth factor receptor HER2 (Her2/neu, ErbB2, or c-erb-b2) is a growth factor receptor that is expressed on many cell types. Cancer cells that over-express HER2 are well known in the art and include, for example, epithelial cancers, such as breast, ovarian, pancreatic, and colorectal carcinomas (Milenic et al., Clinical Cancer Research 10, 7834-7841 (2004)). Other cancer types known to over-express HER2-proteins include salivary gland cancer, stomach cancer, kidney cancer, prostate cancer, and non-small cell lung cancer. See, for example, Mass (Int. J. Radiat. Oncol. Biol. Phys., 58(3): 932-940 (2004)), Wang et al., (Semin. Oncol., 28 (5 Suppl. 16): 115-124 (2001)), and Scholl et al., (Ann. Oncol., 12 (Suppl. 1): S81-S87 (2001)). HER1 is epidermal growth factor receptor (EGFR, ErbB1), which is a cell surface glycoprotein. Cancer cells that over-express HER1 also are well known in the art and include, for example, breast cancer, glioblastoma multiforme, lung cancer, head and neck cancer, ovarian cancer, cervical cancer, bladder cancer, and esophageal cancer. See, for example, Nicholson et al. (Eur. J. Cancer, 37 (Suppl 4): S9-15 (2001)).

In a preferred embodiment, Herceptin™ is the biomolecule in the multifunctional particle that can target epithelial cancer cells.

In an embodiment for studying MS, the biomolecule is an antibody that targets HLA-DR (e.g., L243). In this context, the cells to be imaged can be any cells that express HLA (e.g., HLA-DR). Such cells typically can be found in the brain.

The multidentate ligand can be complexed with a paramagnetic metal atom and used as a relaxation enhancement agent for magnetic resonance imaging. When administered to a mammal (e.g., a human), the multifunctional particle distributes in various concentrations to different tissues, and catalyzes the relaxation of protons in the tissues that have been excited by the absorption of radiofrequency energy from a magnetic resonance imager. This acceleration of the rate of relaxation of the excited protons provides for an image of different contrast when the mammal is scanned with a magnetic resonance imager. The magnetic resonance imager is used to record images at various times, generally either before and after administration of the multifunctional particle, or after administration only, and the differences in the images created by the presence of the multifunctional particle in tissues are used in diagnosis. Guidelines for performing imaging techniques can be found in Stark et al., Magnetic Resonance Imaging, Mosbey Year Book: St. Louis, 1992.

A desirable embodiment of this diagnostic process uses ¹¹¹In and/or ¹⁷⁷Lu. For example, the radioactive probe ¹¹¹In decays with a half life of 2.8 days (67 hours) to an excited state of the daughter nucleus ¹¹¹Cd. From this excited state, a cascade of two gamma-rays is emitted, encompassing an isomeric state with a half life of 85 ns. ¹¹¹In is useful for single photon emission computed spectroscopy (SPECT), which is a diagnostic tool. Thus, when ¹¹¹In (or ¹⁷⁷Lu) is complexed to a multifunctional particle, which can specifically localize in a tumor, then that particular localization can be three-dimensionally mapped for diagnostic purposes in vivo by SPECT. Alternatively, the emission can be used in vitro in radioimmunoassays. In view of the foregoing, the present invention also provides a method for SPECT imaging in a mammal, such as a human. In an embodiment, the method comprises administering to the mammal a multifunctional particle, in which the imaging agent emits a single photon, in an amount effective to provide an image; and exposing the mammal to an energy source, whereupon a SPECT image is obtained.

For purposes of the present invention, mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human. Furthermore, the host can be the unborn offspring of any of the forgoing hosts, especially mammals (e.g., humans), in which case any screening of the host or cells of the host, or administration of compounds to the host or cells of the host, can be performed in utero.

The following examples further illustrate the invention but, of course, should not be construed as in any way limiting its scope.

### Example 1

This example demonstrates a synthesis of ultra-small superparamagnetic iron oxide nanoparticles (USPIOs) in accordance with an embodiment of the invention.

With a stoichiometric ratio of 2Fe³⁺:Fe²⁺, 16 mmol (4.43 g) FeCl₃.6H₂O and 8 mmol (1.625 g) of FeCl₂.4H₂O are dissolved in 190 mL of deionized (DI) water at room temperature by magnetic stirring in a beaker. Under conditions of vigorous stirring, 10 mL of 25% NH₃ is poured down the vortex of the iron solution. Immediately, magnetite forms a black precipitate. The USPIO solution is stirred for ten minutes, followed by three washes with DI water. Washing procedures are performed by putting the solution in a strong magnet, such as an electron paramagnetic resonance magnet, allowing the particles to be pulled to the side by the magnetic field. The clear supernatant is then removed by a pipette. In order to stabilize the particles in solution, the particles are surface-complexed with citrate ions. First, the particle surface is converted from negative to positive by washing twice with 2M HNO₃. These washes with an acid not only reverse the zeta potential of the magnetite colloid and remove any remaining ammonium ions, but also cause the material to release Fe²⁺, converting the magnetite to maghemite, with no reduction in particle size (Jolivet et al., Journal of Colloid and Interface Science 125, 688-701 (1988)).

The leaching of Fe²⁺ is noted by the change in supernatant color to a rusty yellow. After the second wash, the particle solution is diluted to 100 mL with water. Samples at this point are evaluated for zeta potential. In one case, the particles are left in HNO₃ for five days to ensure complete conversion of magnetite to maghemite and then washed and evaluated for zeta potential. Typically, however, the protocol for stabilization with citrate continues by raising the pH to 2.5 with NaOH. While maintaining ˜pH 2.5 with perchloric acid, a volume of 5 mL of 0.5M Na₃[C₃H₅O(COO)₃] solution is added, and the solution is stirred for an hour and a half. The particles are washed with DI water and diluted to 50 mL (˜pH 6). The final citrate-complexed USPIOs are quite stable at this pH because the unadsorbed carboxylate groups of the weakly acidic citrate are deprotonated (Bee et al., Journal of Magnetism and Magnetic Materials 149, 6-9 (1995)):

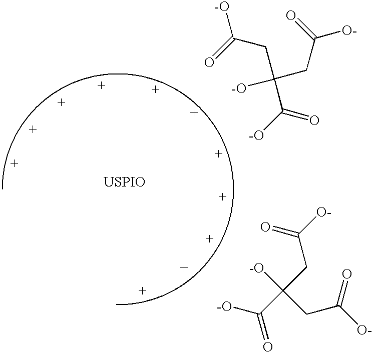

Next, a thin (about 2-5 nm) shell of silica is deposited on the surface of the USPIOs. In a typical synthesis, 30 nmol of USPIO are sonicated in 2.5 mL DI water for ten minutes to ensure even distribution and prevent aggregation. A volume of 250 μL, tetraethylorthosilicate (TEOS) is injected into 2.25 mL of ethanol, and this solution is added to the USPIO solution. To catalyze the reaction, 100 μL of triethylamine is added. The reaction is sonicated for fifteen minutes and then washed by magnetic separation with DI water.

### Example 2

This example demonstrates transmission electron microscopy (TEM) characterization of the USPIOs prepared in Example 1 in accordance with an embodiment of the invention.

Both bare USPIO and silica-coated USPIOs samples are drop-casted on carbon grids. The USPIO core of the particles have an average diameter of 9.2 nm (s=1.4 nm). Using this diameter, the number of USPIOs synthesized in Example 1 is calculated. Assuming the complete precipitation of iron chloride, no losses during washing, and that the particles are spherical, 9.01e17 (1500 nmol) USPIO are produced per batch. In the final volume of 50 mL H₂O, the concentration of USPIO is 30 nmol/mL. TEM measurements of silica layers are used to determine the optimal conditions for the protocol to generate shells of 2 nm thickness.

### Example 3

This example demonstrates a conjugation of Cy5.5 to a USPIO in accordance with an embodiment of the invention.

USPIOs are first coated with silica and then conjugated to Cy5.5 using a known method. Instead of functionalizing particles with APTES and then adding Cy5.5, first APTES should be attached to Cy5.5. Then the APTES-Cy5.5 conjugate can react with the silica surface of particles. The Cy5.5-silica-USPIO particles are coated with a final layer of silica to encapsulate the dye and make the outer surface of the particles biocompatible. The same silication protocol is used with a shortened reaction time. Samples from each point during nanoparticle synthesis are observed using transmission electron microscopy (TEM), confirming that the Cy5.5 conjugation process did not degrade the silica layer.

Thin layer chromatography (TLC), a technique used to for separating organic compounds, is used to confirm conjugation in the APTES-Cy5.5 sample. A silica plate is dotted with the appropriate samples and the bottom edge is placed in a reservoir of 20% methanol in chloroform. The solvent moves up the plate by capillary action. When the solvent front reaches the other edge of the plate, it is removed. The separated spots are visualized with ultraviolet light and by placing the plate in iodine vapor. The less-polar conjugate moves off the polar silica plate earlier and travels significantly farther than the more polar Cy5.5 (—NHS ester). R_{f} values of Cy5.5, APTES-Cy5.5, and APTES are 0.36, 0.49, and 0.03, respectively. The conjugate moves 38% farther than Cy5.5, confirming conjugation.

### Example 4

This example demonstrates a conjugation of an antibody to a USPIO in accordance with an embodiment of the invention. See FIG. 2.

Antibody is prepared by incubating in 1× phosphate buffered saline (PBS) at room temperature with s-SMCC at a molar ratio of 10:1 s-SMCC:Ab for 1.5 hours with gentle stirring. The activated Ab is separated from excess linker by spinning at 3000 g/10° C. in a Centriprep filter with a molecular weight cutoff (MWCO) of 10,000.

USPIOs are synthesized according to Examples 1-3, and further functionalized with thiols. For a typical conjugation, 5 nmol USPIOs are dispersed in 5 mL ethanol and incubated in room temperature with 250 μL (3-mercaptopropyl)trimethoxysilane (MPS) for forty minutes.

The particles are then washed by magnetic separation into a solvent of PBS. The maleimide-activated Ab and thiol-activated USPIOs are then allowed to react overnight in 4° C. For the final step, ethylmaleimide is added to cap any free thiols. The Ab-USPIO sample is washed by magnetic separation with PBS and stored in 4° C.

### Example 5

This example demonstrates an in vitro study with L243-conjugated nanoparticles (NPs) in accordance with an embodiment of the invention.

Samples of one million L cells that express L243 receptors are incubated for thirty minutes in room temperature with 0.001 nmol of L243-conjugated or only thiol-functionalized NPs. Free thiols were not capped with ethylmaleimide. L243 labeled with the fluorophore PE is used to stain with as a positive control. The cells are then washed three times with 4% FBS/PBS by centrifuging at 500 g/4° C. for two minutes and decanting the media. The samples are diluted to 1 mL 4% FBS/PBS and analyzed with flow cytometry using an APC laser which excites at 630 nm and collects emissions at 660 nm. The cells are gated as R1 and 10,000 counts are collected from each sample. The percentage of cells that display fluorescence is recorded and signal-to-noise ratio calculated by dividing percentage fluorescence of NP-L243 stained cells by NP—SH stained cells.

The results show that the antibody-conjugated NPs are successful in staining cells in vitro with a signal-to-noise ratio of 12.5. This ratio is not as high as the control ratio of 47.7, but it is necessary to note that the filter being used is not optimal for Cy5.5 emissions, whereas for the positive control a PE filter, specific to the fluorophore, is used.

### Example 6

This example demonstrates an in vitro study with Herceptin™-conjugated NPs in accordance with an embodiment of the invention.

NPs are conjugated to Herceptin™ and a negative mAb, HuM195. To reduce oxidation of thiols, the reactions are conducted under argon bubbling Argon is a larger molecule than oxygen and so it displaces any oxygen in the solution. The number of free thiols per particle before and after antibody conjugation are quantified using Ellman's reagent. When 5,5′-dithio-bis-(2-nitrobenzoic acid), more commonly known as DTNB or Ellman's reagent, is reduced by free thiols, it releases 2-nitro-5-thiobenzoic acid (TNB) as a product that can be detected by absorbance at 412 nm (Ellman, Arch. Biochem. Biophys 82, 70-77 (1959)). The results from the Ellman's test across various samples showed that these particle samples have free thiols and approximately a third are either oxidized or attached to Ab after conjugation but before being capped by ethylmaleimide.

A Lowry protein determination assay (Lowry et al., J Biol Chem 193, 265-275 (1951)) shows protein conjugation. Typical Herceptin™:NP reaction ratios yielded ˜7 Herceptin™ per particle.

For these studies, SKOV cells that express the Herceptin™ receptor HER2 were used for staining. SKOV cells are stained and analyzed with flow cytometry. Herceptin™ and HuM195 conjugated directly to Cy5.5 are used as controls. The stains show a high 20.9 signal-to-noise ratio for the conjugated particles. The 20.9 signal-to-noise ratio is significantly higher than the controls (5.4) and shows that the Ab-conjugation is successful at targeting the particles.

### Example 7

This example demonstrates antibody chelation to ¹¹¹In in accordance with an embodiment of the invention.

For demetallation of all buffers, a Chelex-100 (BioRad Na⁺ form 200-400 mesh resin) column is used. Two buffers are prepared:
1) 10× Conjugation Buffer: 80.44 g NaHCO₃, 4.50 g Na₂CO₃, and 175.32 g NaCl in 2 L deionized water; and
2) 10× Ammonium Acetate Buffer: 1.5M NH₄OAc solution
   and passed through the chelex column to remove any metal. Glass containers are avoided and only metal free pipette tips are used. Extreme care is taken to keep all steps metal-free. To a 5.4 mL sample of 5 mg/mL Herceptin™ in PBS, 595 μL of 10× conjugation buffer is added, making it 1×. 60 μL of 0.5M/pH8.0 ethylenediaminetetraacetic acid (EDTA) is added to remove any free metals in the solution. A mass of 1.8 mg (10×mols) of chelate CHX-A″ is reacted with the mAb solution in 37° C. for 3.5 hours. Subsequently, the reaction mixture is dialyzed (SPECTRUM cellulose dialysis kit, MWCO 10 000) five times against 1 L metal-free 1× ammonia acetate buffer for a minimum of four hours each at 4° C. while stirring gently. The number of chelates per mAb (2.265 chelates per Herceptin™) is evaluated by the Lowry assay and a spectrophotometric assay using yttrium-arsenazo III complex at 652 nm (Pippin et al., Bioconjugate Chemistry 3, 342-345 (1992)).

Typically, to label the chelated Herceptin™ with ¹¹¹In, 1.0 mCi would be incubated at 37° C. with 100 mg mAb for half an hour. A volume of 5 μL of 0.5M EDTA can be injected to remove free ¹¹¹In and then the solution can be collected in fractions as it is passed through a PD10 desalting column with PBS solvent. The first peak of radioactive material collected would be the labeled antibody.

### Example 8

This example demonstrates chelation of the antibody cetuximab to the multidentate ligand CHX-A″ and subsequent conjugation to a SCION particle in accordance with an embodiment of the invention. See FIG. 4B.

Only demetallated buffers are used during this entire conjugation. A Chelex-100 (BioRad Na⁺ form 200-400 mesh resin) column can be used to remove metals. Two buffers are prepared:
1) 10× Conjugation Buffer: 80.44 g NaHCO₃, 4.50 g Na2CO3, and 175.32 g NaCl in 2 L deionized water
2) 10× Ammonium Acetate Buffer: 1.5M NH4OAc solution
   and passed through the chelex column. Glass containers are avoided and only metal free pipette tips used. Extreme care is taken to keep all steps metal-free.

To prepare cetuximab for chelation, the antibody is washed into 1× conjugation buffer and 50 mM EDTA in PBS and warmed in a 37° C. water bath for ten minutes. The concentrated antibody solution (10 mg/mL) is then reacted with the chelate CHX-A″ at a molar ratio of 1:10 in 37° C. on a shaker for 3.5 hours. Subsequently, the reaction mixture is dialyzed (SPECTRUM cellulose dialysis kit, MWCO10000) six times against 1 L metal-free 1× ammonia acetate buffer for a minimum of four hours each at 4° C. while stirring gently. The number of chelates per mAb (1.9 chelates per cetuximab) is evaluated by the Lowry assay and a spectrophotometric assay using yttrium-arsenazo III complex at 652 nm.

Using centrifugation with a 50000MWCO spin filter, chelated cetuximab is concentrated into metal-free thiolation buffer (5 mM EDTA in PBS buffer, pH 8.0). The 10 mg/mL antibody solution is then reacted with Traut's reagent at a 1:15 molar ratio for one hour in room temperature, capped with argon, and on a rotator. These conditions are determined to yield 1.8 —SH groups per cetuximab molecule. Excess Traut's reagent is removed by passage of the reaction solution through a PD-10 column eluted with PBS buffer. The —SH concentration is measured using Ellman's reagent.

NPs as prepared by Examples 1-3 and that are functionalized with maleimido groups are stored in PBS at a concentration of 1 nmol/mL NPs. Thiolized and chelated cetuximab is reacted with the particle solution while capped under argon for 1 hr in room temperature on a rotator and then overnight in 4° C. Excess free SH groups are capped with excess iodoacetamide solution by reacting in room temperature for 1.5 hr. Finally, the reaction mixture is dialyzed into PBS buffer at 4° C. with 4 buffer changes over 48 hours.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A multifunctional particle comprising:
(a) an inner metallic core,
(b) a biocompatible shell comprising an optical contrast agent embedded therein, and
(c) a targeting biomolecule conjugated to the biocompatible shell and a multidentate ligand, wherein the multidentate ligand is chelated to an imaging agent.

2. The multifunctional particle of claim 1, wherein the diameter of the inner metallic core is less than about 50 nm.

3. The multifunctional particle of claim 1, wherein the inner metallic core is magnetic.

4. The multifunctional particle of claim 1, wherein the inner metallic core comprises superparamagnetic iron oxide.

5. The multifunctional particle of claim 4, wherein the inner metallic core comprises maghemite/magnetite (γ-Fe₂O₃/Fe₃O₄).

6. The multifunctional particle of claim 1, wherein the biocompatible shell comprises a first innermost layer in contact with the inner metallic core and a second outermost layer.

7. The multifunctional particle of claim 6, wherein the first innermost and second outermost layers of the biocompatible shell are of the same material.

8. The multifunctional particle of claim 1, wherein the biocompatible shell comprises silica.

9. The multifunctional particle of claim 6, wherein the first innermost and second outermost layers of the biocompatible shell are of different materials.

10. The multifunctional particle of claim 1, wherein the optical contrast agent is selected from the group consisting of a cyanine dye, rhodamine, coumarin, pyrene, dansyl, fluorescein, fluorescein isothiocyanate, carboxyfluorescein diacetate succinimidyl ester, an isomer of fluorescein, R-phycoerythrin, tris(2′,2-bipyridyl)dichlororuthenium(II) hexahydrate, Fam, VIC®, NED™, ROX™, calcein acetoxymethylester, DiIC₁₂, and anthranoyl.

11. The multifunctional particle of claim 1, wherein the targeting biomolecule is an antibody.

12. The multifunctional particle of claim 11, wherein the antibody is selected from a group consisting of scFv, F(ab′)₂, and F_{ab}.

13. The multifunctional particle of claim 1, wherein the targeting biomolecule is a peptide or protein.

14. The multifunctional particle of claim 1, wherein the imaging agent is a radioisotope.

15. The multifunctional particle of claim 1, wherein the imaging agent is a gamma-emitting radioisotope.

16. The multifunctional particle of claim 1, wherein the imaging agent is a radioactive lanthanide.

17. The multifunctional particle of claim 1, wherein the imaging agent is selected from the group consisting of ⁸⁶Y, ⁶⁴Cu, ⁸⁹Zr, ¹²⁴I, ⁶⁶Ga, ⁶⁸Ga, ⁶⁷Ga, ¹²³I, ²⁰³Pb, and ¹¹¹In.

18. The multifunctional particle of claim 1, wherein the targeting biomolecule binds to a receptor on the surface of a cancer cell.

19. The multifunctional particle of claim 11, wherein the antibody targets HER2 or HLA-DR.

20. (canceled)

21. A composition comprising (a) at least one multifunctional particle of claim 1; and (b) a carrier.

22. The composition of claim 21, wherein the carrier is pharmaceutically acceptable.

23. A method of imaging a cancer cell in a mammal comprising
(a) administering to the mammal intravenously the multifunctional particle of claim 1;
(b) contacting a cancer cell surface receptor with the targeting biomolecule of the particle;
(c) observing a fluorescence emission from the optical contrast agent or detecting an emission from the imaging agent of the particle by spectroscopy.

24. The method of claim 23, wherein the spectroscopy is selected from the group consisting of single photon emission computed spectroscopy (SPECT), positron emission tomography (PET), gamma scintigraphy, and magnetic resonance imaging (MRI).

25. The method of claim 23, wherein the cancer cell over-expresses HER 1 and/or HER2.

26. The method of claim 23, wherein the cancer cell is an epithelial cancer cell.

27. The method of claim 26, wherein the epithelial cancer cell is breast carcinoma, ovarian carcinoma, pancreatic carcinoma, or colorectal carcinoma.

28. A method for obtaining a diagnostic image of a mammal comprising
(a) administering to the mammal the multifunctional particle of claim 1, in an amount effective to provide an image; and
(b) exposing the mammal to an energy source, whereupon a diagnostic image of the mammal is obtained.

29. The method of claim 28, wherein the diagnostic image is magnetic resonance image (MRI), an x-ray contrast image, single photon emission computed spectroscopy (SPECT) image, or a positron emission tomography (PET) image.
